(19) United States
(12) Patent Application Publication
(10) Pub. No.: US 2025/0305005 A1
(43) Pub. Date: Oct. 2, 2025

(54) CARBON DIOXIDE PROCESSING IN GAS-OIL SEPARATION PLANT

(71) Applicant: SAUDI ARABIAN OIL COMPANY, Dhahran (SA)

(72) Inventors: Manal Faisal ALQAHTANI, Thuwal (SA); Nouf ALJABRI, Thuwal (SA); Ameerah M. BOKHARI, Thuwal (SA)

(73) Assignee: SAUDI ARABIAN OIL COMPANY, Dhahran (SA)

(21) Appl. No.: 18/616,485

(22) Filed: Mar. 26, 2024

(51) Int. Cl. C12P 5/02; C12P 1/00; C12P 7/06; C12P 7/40; C25B 1/135

(52) U.S. Cl. CPC C12P 5/023; C12P 1/00; C12P 7/06; C12P 7/40; C25B 1/135

(57) ABSTRACT

Microbial electrosynthesis (MES) may be used in converting carbon dioxide from a gas-oil separation plant (GOSP) to fixed carbon products. Example methods of MES may include: separating emitted carbon dioxide from an extracted gas-oil in a GOSP; dispersing the emitted carbon dioxide in a first portion of electrolyte solution in a cathode chamber of an electrolysis reactor; converting the emitted carbon dioxide to a fixed carbon product with a biocatalyst within the cathode chamber; and maintaining an electric potential between a cathode of the cathode chamber and an anode of an anode chamber with a potentiostat, wherein the cathode chamber and the anode chamber are separated by a semi-permeable membrane.

### FIELD OF THE DISCLOSURE

The present disclosure relates generally to operation of a gas-oil separation plant (GOSP) and, more particularly, to processing of exhaust byproducts of a GOSP.

### BACKGROUND OF THE DISCLOSURE

Crude oil produced from a subterranean wellbore often contains hydrocarbons mixed with impurities such as water and suspended solids. The crude oil may be separated into its constituent components at a GOSP facility near the wellbore such that the unwanted components do not need to be transported further. The hydrocarbons (oil and associated gases) may be separated from the water, and the resulting fluid streams may be directed to individual locations for further processing.

Conventional GOSP facilities may suffer from deficiencies including low product yield, inefficient use of available heat sources (e.g., discharge streams of compressors), many separate units being used to meet desired basic sediment and water (BS&W) specifications, high operating costs due to heating requirements, a large spatial footprint and high capital costs. By integrating and simultaneously implementing other processes into a GOSP facility, and by effectively identifying byproducts of the GOSP facility that may prove to be valuable resources, more efficient processes and systems may be defined or provided.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

A nonlimiting example method of the present disclosure includes: separating emitted carbon dioxide from an extracted gas-oil in a gas-oil separation chamber of a gas-oil separation plant (GOSP); dispersing the emitted carbon dioxide in a first portion of electrolyte solution in a cathode chamber of an electrolysis reactor; converting the emitted carbon dioxide to a fixed carbon product with a biocatalyst within the cathode chamber; and maintaining an electric potential between a cathode of the cathode chamber and an anode of an anode chamber with a potentiostat, wherein the cathode chamber and the anode chamber are separated by a semi-permeable membrane.

A nonlimiting example system of the present disclosure includes: a separation chamber of a gas-oil separation plant (GOSP), wherein the separation chamber is configured to separate emitted carbon dioxide from an extracted gas-oil; a GOSP carbon dioxide line for conveying the emitted carbon dioxide; a reaction chamber that contains therein electrolyte solution, wherein the electrolyte solution comprises an aqueous fluid, and wherein the reaction chamber includes therein: a cathode chamber, wherein the cathode chamber contains therein a first portion of the electrolyte solution, wherein the cathode chamber has a cathode at least partially immersed in the first portion of the electrolyte solution, wherein the cathode chamber is fluidly connected to the GOSP carbon dioxide line such that the emitted carbon dioxide is at least partially dispersed within the first portion of the electrolyte solution, wherein the cathode chamber has a biocatalyst therein, and wherein the biocatalyst is capable of converting the emitted carbon dioxide to a fixed carbon product; an anode chamber, wherein the anode chamber contains therein a second portion of the electrolyte solution, wherein the anode chamber has an anode at least partially immersed in the second portion of the electrolyte solution; a semi-permeable membrane separating the cathode chamber and the anode chamber, wherein the semi-permeable membrane conveys; and a potentiostat, wherein the potentiostat is electrically connected to the cathode and the anode, and wherein the potentiostat maintains an electric potential across the cathode and the anode.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a schematic flow diagram of an example GOSP system according to the present disclosure.

FIG. 2. is a diagram of an example MES unit according to the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to operation of a gas-oil separation plant (GOSP) and, more particularly, to processing of exhaust byproducts of a GOSP.

Carbon dioxide may be captured from the exhaust of a GOSP facility, including, but not limited to, for example, from a combustion gas turbine of the GOSP. Subsequently, carbon dioxide emitted from the GOSP may be converted to fixed carbon products useful in a variety of industries through use of a biocatalyst in a microbial electrosynthesis (MES) reaction. Examples of fixed carbon products may include, but are not limited to, fuels (e.g., ethanol, methane), organic acids, polymers, and the like. In this manner, carbon dioxide emissions overall for the GOSP facility may be reduced, an overall efficiency of the GOSP facility may be increased and a profit may be generated from exported fixed carbon products. Furthermore, according to the present disclosure, the carbon dioxide may be processed immediately or near immediately upon emission from the GOSP, thus resulting in reduced associated emissions resulting from transport and storage of emitted carbon dioxide that would conventionally undergo further delayed processing.

“Immediately” or “near immediately,” and grammatical variations thereof as used herein may include wherein carbon dioxide is delivered to the MES unit within 5 minutes (or 10 minutes, or 1 minute) of separation from gas-oil in the GOSP.

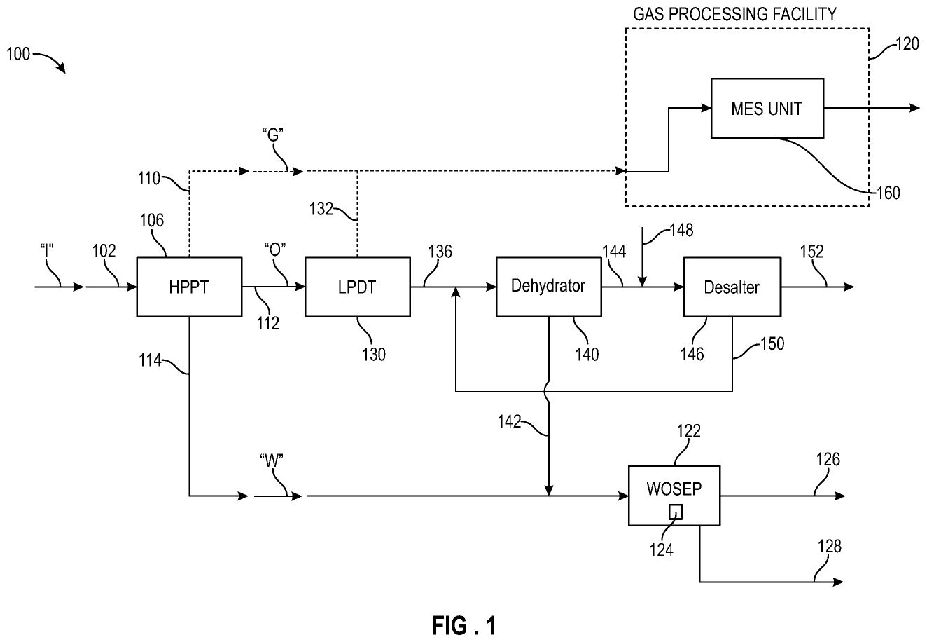

FIG. 1 is a schematic flow diagram of an example GOSP system 100 including a crude inlet line 102. The crude inlet line 102 may be a transmission pipeline that carries an inlet fluid stream “I” from the wellhead(s) of one or more hydrocarbon-producing wellbores (not shown) to the GOSP system 100. The inlet fluid stream “I” may include a mixture of hydrocarbon gases, liquid oil, water (in both liquid and vapor forms), and salt and other solid sediments. It is important that at least a particular amount or proportion of the water, salt and sediments be removed from the inlet fluid stream “I” in order to prepare the fluid inlet steam “I” for further processing at a refinery and to avoid the corrosion of downstream piping, fittings, instrumentation and the like.

The inlet fluid stream “I” passes through the crude inlet line to a high-pressure production trap (HPPT) 106. The HPPT 106 may include a separator vessel such as a horizontal, three-phase separator, which generally uses gravity to separate the inlet fluid stream “I” into a gas component, an oil component and a water component. In some embodiments, the separation vessel may alternatively or additionally employ various other methods of separating the incoming inlet fluid stream “I” into components including impingement, changing a flow direction and/or velocity of the fluid stream and/or application of a centrifugal force. The gas component exits the HPPT 106 through a gas line 110 as gas stream “G,” the oil component exits the HPPT 106 through an oil line 112 as an oil stream “O” and the water component exits the HPPT 106 through a water line 114 as a water stream “W.”

The gas stream “G” may be directed to a gas processing facility 120 for gas recovery in accordance with various aspects of the present disclosure as described in greater detail below. Transferring gas stream “G” to gas processing facility 120 may include processing of emitted carbon dioxide to one or more fixed carbon products within microbial electrosynthesis (MES) unit 160 of gas processing facility 120. The water stream “W” may be directed to a water-oil separator (WOSEP) 122, which may have an internal separator 124 (e.g., a weir arrangement) that separates any remaining crude oil in the water stream “W.” The WOSEP 122 may have a first water discharge 126 through which water with a first oil content may be discharged. For example, water having less than 1 vol % of crude oil, less than 0.1 vol % of crude oil, or less than 0.01 vol % of crude oil may be discharged through the first water discharge 126 for disposal, injection or other uses. The WOSEP may have a second water discharge 128 through which water with a second oil content, e.g., water having crude oil in a range of about 1 vol % to about 10 vol %, may be discharged. Water discharged through the second water discharge 128 may be directed through further processing units (not shown) for recovery of the retained oil.

The oil stream “O” exiting the HPPT 106 may be directed to a low pressure degassing tank (LPDT) 130. The LPDT 130 may include a cyclonic separator or other mechanisms for further separating entrained gas from the oil stream “O.” The gas separated in the LPDT 130 may optionally, in some embodiments, pass through a gas line 132 to join the gas stream “G” in the gas line 110. The remaining oil stream “O” may exit the LPDT 130 through oil line 136, which carries the oil stream to a dehydrator 140.

As discussed above, gas in gas stream “G”, including emitted carbon dioxide gas, transferred to the gas processing facility 120 may originate from a separation chamber (e.g., an HPPT, an LPDT) of the GOSP. In some embodiments, upon entering the gas processing facility 120, gas stream “G” may be suitably separated into various components or otherwise initially processed. Subsequently, emitted carbon dioxide from the GOSP included in the gas stream “G” may be processed through MES methods in MES unit 160.

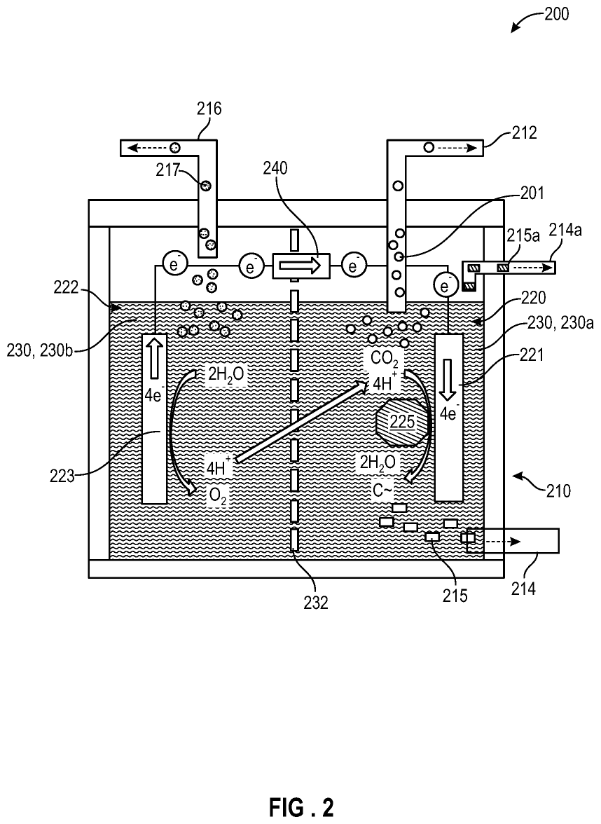

MES systems and methods may include an MES unit. A nonlimiting example MES unit 200 for processing emitted carbon dioxide 201 according to the present disclosure is shown in FIG. 2. The unit 200 includes a reaction chamber 210 having therein an electrolyte solution 230. The reaction chamber 210 may have a cathode chamber 220 with a cathode 221 therein and an anode chamber 222 having an anode 223 therein. The cathode 221 may be immersed in a first portion 230a of electrolyte solution 230 and the anode 223 may be immersed in a second portion 230b of electrolyte solution 230. Furthermore, cathode chamber 220 may have a biocatalyst 225 therein. Biocatalyst 225 (described in further detail below) may be present in cathode chamber 220 in any suitable form. For example, biocatalyst 225 may be dispersed within the first portion 230a of electrolyte solution, may exist as a layer attached to cathode 221, may exist on a support structure, the like, or any combination thereof.

Reaction chamber 210 may have one or more inlets (illustrated as a single inlet 212) for transferring emitted carbon dioxide 201 to reaction chamber 210, specifically to cathode chamber 220 therein. Inlet 212 may be connected to a GOSP carbon dioxide line or any other such line for conveying the emitted carbon dioxide 201 from a GOSP (e.g., from a separation unit of a GOSP) to the MES unit.

Reaction chamber 210 may additionally have one or more outlets. In the illustrated example, the one or more outlets include a products outlet 214 for removing fixed carbon products 215 from cathode chamber 220, and a first gas outlet 216 for removing oxygen gas 217 or any other such gas from anode chamber 222. In some examples, the one or more outlets may optionally or in the alternative include a second gas outlet 214a for removing gaseous fixed carbon products 215a (e.g., methane) from cathode chamber 220.

Reaction chamber 210 may furthermore have a semi-permeable membrane 232 (described in more detail below) fluidly separating the first portion of electrolyte solution 230a of the cathode chamber 220 and the second portion 230b of electrolyte solution of the anode chamber 222.

Reaction chamber 210 may have a potentiostat 240 electrically connected to the cathode 221 and the anode 223, such that the potentiostat maintains an electric potential across the cathode 221 and the anode 223. It should be noted that potentiostat 240 may be located internally within reaction chamber 210 (as shown in FIG. 2) or potentiostat 240 may be external to reaction chamber 210.

Reaction chamber 210 may be constructed of any suitable material and may be of any suitable size or configuration.

Continuing to describe FIG. 2, the diagram additionally shows a nonlimiting example reaction mechanism for the production of fixed carbon products from carbon dioxide. The mechanism described herein is not to be bound by theory, and additionally it should be noted that other mechanisms not depicted in FIG. 2 may exist for production of fixed carbon products from carbon dioxide in accordance with the present disclosure. Carbon dioxide (CO₂) 201 may enter cathode chamber 220 through inlet 212 and be dispersed within the first portion 230a of electrolyte solution. CO₂ 201 may accept 4 electrons (4e⁻) from cathode 221 and be catalyzed along with 4H⁺ ions by biocatalyst 225 to form 2 water molecules (2H₂O) as well as fixed carbon product 215 (also depicted as “C˜”). C˜ 215 may subsequently exit cathode chamber 220 through products outlet 214. Optionally or in the alternative, gaseous fixed carbon products 215a may subsequently exit cathode chamber 220 through second gas outlet 214a. The 4 electrons (4e⁻) may be transferred to the cathode 221 by the potentiostat 240 and wiring connected thereto. At the anode 223, 2 water molecules (2H₂O) may be split, generating 4e⁻ as well as 4H⁺, and oxygen (O₂) 217. O₂ 217 may subsequently exit anode chamber 222 through first gas outlet 216. The reactions shown in FIG. 2 and described above are additionally summarized in Equations 1 and 2 below, wherein Equation 1 may occur at a cathode and Equation 2 may occur at an anode. It should be noted that in some embodiments, Equation 2 may be optional.

CO₂+4e⁻+4H⁺→C˜+2H₂O Equation 1

2H₂O→O₂+4e⁻+4H⁺ Equation 2

Optionally or in the alternative of Equation 1, the cathode may include one or more of reactions depicted in Equations 3-5, wherein the fixed carbon product comprises a gaseous fixed carbon product comprising methane (CH₄).

8H⁺+8e⁻→4H₂ Equation 3

CO₂+4H₂→CH₄+2H₂O Equation 4

CO₂+8H⁺+8e⁻→CH₄+2H₂O Equation 5

It should be noted that additional reactions not shown or described may contribute to the conversion of carbon dioxide to fixed carbon product or otherwise be present in the above-described system. It should also be noted that variations of the above nonlimiting reaction may occur in accordance with the present disclosure, depending on the chemical makeup of fixed carbon products being produced.

### Fixed Carbon Products

Fixed carbon products of relevance in the present disclosure may include any classes of carbon molecules capable of being produced by microbial electrosynthesis (MES) with carbon dioxide as a reactant. Examples of fixed carbon products may include, but are not limited to, alkanes (e.g., methane, ethane, propane, the like), alkyl alcohols (e.g., methanol, ethanol, isopropanol, the like), carboxylic acids (e.g., formic acid, acetic acid, propionic acid) and/or derivatives thereof (e.g., acetates, formate, caproate, lactate, valerate, butyrate), polymers (e.g., polyhydroxyalkanoate (PHA) ethanol), the like, or any combination thereof.

Types and quantities of fixed carbon products produced by MES according to the present disclosure may be influenced by factors including, but not limited to, for example, inoculum source(s), enriched communities, substrate type, electrode materials, reaction conditions (e.g., temperature, pressure, the like), the like, or any combination thereof).

Fixed carbon products comprising methane (CH₄) may occur through reduction of carbon dioxide by methanogenic microorganisms such as those of the genus Methanobacterium. Fixed carbon products comprising acetate (C₂H₄O₂) may be generated by acetogenic bacteria such as those of the genus Clostridium. Furthermore, various other strains of microorganisms may allow for production of other fixed carbon products under distinct reaction conditions, the like, or any combination thereof. Various microorganisms responsible for producing fixed carbon products may, in some embodiments, additionally produce hydrogen (H₂).

Without being bound by theory, the production of fixed carbon products (e.g., CH₄) through MES may generally not be thermodynamically favorable and may require external energy input to drive the overall reaction. Microorganisms (e.g., methanogens) can generate fixed carbon products (e.g., CH₄) through two mechanisms: direct Extracellular Electron Transfer (EET) or indirect EET. Direct EET allows for direct uptake of electrons from an electrode to reduce CO₂ and generate fixed carbon products. Direct EET may, in some embodiments, occur at a cathode potential of about −0.24 V (vs. a Standard Hydrogen Electrode (SHE)). Equations 1 and/or 5 (shown above) show nonlimiting examples of direct EET.

Indirect EET may allow for intermediate hydrogen generation and subsequent CO₂ therefrom. Intermediate hydrogen generation may, in some embodiments, occur at a cathode potential of about −0.41 V (vs. SHE). Intermediate hydrogen generation may occur either abiotically via a Hydrogen Evolution Reaction (HER) or via a biotic HER. A nonlimiting example of abiotic HER is shown in Equation 3. After occurrence of either biotic and/or abiotic HER, a biocatalyst (e.g., a hydrogenotrophic methanogen) on the surface of a cathode may utilize hydrogen generated as a source of reducing equivalents for CO₂ reduction to a fixed carbon product (e.g., CH₄). Equation 4 (shown above) shows a nonlimiting example of use of hydrogen generated for CO₂ reduction to a fixed carbon product.

### Biocatalyst

Biocatalysts relevant to the present disclosure may comprise any suitable biological means of microbial electrosynthesis (MES) with carbon dioxide. Examples of types of organisms (e.g., microorganisms) suitable for use as biocatalysts may include, but are not limited to, bacteria, archaea, fungi, algae, the like or any combination thereof. Suitable biocatalysts may comprise a bacteria, including a suitable acetogenic bacteria. Examples of suitable bacteria may include, but are not limited to, for example, a bacteria of the genus Sporomusa, Clostridium, Moorella, Geobacter, Anaeromyxobacter, the like, or any combination thereof.

Suitable biocatalysts may comprise methanogenic archaea, such as Methanobacterium and Methanosarcina acetivorans. Such methanogenic archaea may be engineered to utilize electrons from electrodes for carbon dioxide reduction to a specific fixed carbon product such as, for example, methane. Suitable biocatalysts may comprise Sporopomusa ovata. Suitable Sporopomusa ovata may be engineered to yield specific fixed carbon products such as, for example, acetate, ethanol, or the like. Suitable biocatalysts may comprise Clostridium. Suitable Clostridium may be engineered to yield specific fixed carbon products such as, for example, acetate. Suitable biocatalysts may include yeast species such as Saccharomyces cerevisiae. Suitable Saccharomyces cerevisiae may be engineered to yield fixed carbon products such as, for example, terpenoid(s) with greater than 20 carbon atoms. Suitable biocatalysts may comprise microalgae.

Biocatalysts may be engineered to target yield of specific fixed carbon products including, but not limited to, for example, production of key enzymes (e.g., hydrogenase enzymes, carbon fixation enzymes), increasing and/or decreasing catalytic activity of specific metabolic pathways, the like, or any combination thereof. It should be noted that other microorganisms in addition to those described herein may serve as biocatalysts in any combination.

Without being bound by theory, biocatalysts of the present disclosure may interact with carbon dioxide so as to enable carbon dioxide to act as an electron acceptor, converting the carbon dioxide to a fixed carbon product. Biocatalysts of the present disclosure may additionally be present in combination with a mediator (e.g., an electron mediator). Use of mediators may allow for increasing efficiency of electrochemical CO₂ reduction by facilitating electron transfer between an electrode surface and microbial cells. Naturally occurring mediators may contribute to direct EET and/or indirect EET by increasing efficiency thereof. Naturally occurring mediators may include enzymes, redox-active components (e.g., quinones, flavins, and the like), the like or any combination thereof. Alternatively, mediators may include conductive materials such as graphene, carbon nanotubes, poly pyrrole polymers, redox mediators, the like, or any combination thereof. Conductive materials may contribute to direct EET and/or indirect EET by increasing efficiency thereof.

Continuing to not be bound by theory, a nonlimiting example of use of a mediator may include bioanode oxidation reactions facilitated by Geobacter sulfurreduces and/or Shewanella oneidensis. Such bioanode reactions may utilize large multiheme c-type cytochromes as mediators for efficient EET. Additionally, such bioanode reactions may utilize redox-active species such as riboflavin or various trace minerals as mediators in order to increase reaction efficiency. One of ordinary skill in the art will be able to evaluate various mediators for conversion of carbon dioxide to fixed carbon products.

Biocatalysts of the present disclosure may exist in any suitable form including, but not limited to, for example, dispersed within the first portion of electrolyte solution within the cathode chamber, as a layer (e.g., a biofilm) attached to a cathode, on a support structure (e.g., a pellet, a matrix, the like), the like, or any combination thereof.

### Electrodes

Any number of suitable materials can be used in accordance with the present disclosure for cathode and anode electrodes. For example, electrode materials of the present disclosure may include, but are not limited to, carbonous material (e.g., carbon paper, carbon cloth, carbon wool, carbon foam, graphite, graphene, carbon nanotubes, carbon fibers, the like, or any combination thereof), a metal (e.g., platinum, palladium, titanium, gold, silver, copper, iron, tungsten, cobalt, steel, nickel, tin, the like, or any combination thereof), a conductive polymer, the like, or any combination thereof.

### Semi-Permeable Membrane

MES units of the present disclosure may include a semi-permeable membrane that may comprise a proton-exchange membrane in the interior of the reaction chamber, separating fluid between a cathode chamber and an anode chamber. The proton-exchange membrane may be an ion exchange membrane that only permits one-way proton communication between the anode and the cathode. The present disclosure may utilize any suitable proton-exchange membrane. Examples of suitable proton-exchange membrane materials may include, but are not limited to, perfluorinated polymer membranes (e.g., NAFION, available from Chemours).

### Electrolyte Solution

The electrolyte solutions described above may comprise an aqueous fluid (e.g., water) and any suitable electrolyte salt. Suitable electrolyte salts may include, but are not limited to, potassium chloride (KCl), potassium iodide (KI), sodium chloride (NaCl), ammonium chloride (NH₄Cl), potassium sulfonate (K₂SO₄), sodium sulfonate (Na₂SO₄), ammonium sulfonate ((NH₄)₂SO₄), the like, or any combination thereof. The electrolyte salts may be present in any suitable concentration, including, for example, from 0.0001 mol/L (M) to 10 M (or 0.01 M to 10 M, or 0.01 M to 5 M, or 0.01 M to 2 M, or 0.01 M to 1 M, or 0.1 M to 10 M, or 0.1 M to 5 M, or 0.1 M to 2 M, or 0.1 M to 1 M, or 1 M to 5 M, or 1 M to 2 M, or 0.01 M to 0.1 M, or 0.001 M to 0.1 M, or 0.0001 M to 0.1 M, or 0.1 M or less, or 0.01 M or less). It should be noted that compositions and concentrations of electrolyte solutions of the present disclosure may be tailored to the specific microorganisms used for biocatalysis as described above, and additionally may be tailored for desired fixed carbon products being produced. It should further be noted that the electrolyte solutions may comprise gasses (e.g., H₂, CO₂, the like) or various ions (e.g., O₂⁺, CO₂⁺, the like) dissolved therein, in any suitable concentration.

Aqueous fluids for use in electrolyte solutions of the present disclosure may include any suitable aqueous fluid including fresh water, salt water, brine, produced water, or the like. It should be noted that additional water, electrolytes, or other materials may be added to the reaction chamber to offset compositional changes to electrolyte solutions therein when reactants (e.g., carbon dioxide) are added to respective chambers (e.g., anode chamber or cathode chamber) of the reaction chamber and/or when products (e.g., fixed carbon products, oxygen, the like) are removed from respective chambers within the reaction chamber, as described herein.

### Integrated System

It should be noted that one or more MES units described herein may be operated in any suitable manner, including any suitable configuration (e.g., in parallel, in series, the like, or a combination thereof) and including any suitable operational fashion (e.g., a continuous fashion, a batch-wise fashion, the like, or a combination thereof).

For the purpose of these simplified schematic illustrations and description, there may be additional valves, lines, pumps, sensors, controllers, wires, and the like that are customarily employed in GOSP operations that are well known to those of ordinary skill in the art that are not shown.

### ADDITIONAL EMBODIMENTS

Embodiments disclosed herein include:

Embodiment 1: A method comprising: separating emitted carbon dioxide from an extracted gas-oil in a gas-oil separation chamber of a gas-oil separation plant (GOSP); dispersing the emitted carbon dioxide in a first portion of electrolyte solution in a cathode chamber of an electrolysis reactor; converting the emitted carbon dioxide to a fixed carbon product with a biocatalyst within the cathode chamber; and maintaining an electric potential between a cathode of the cathode chamber and an anode of an anode chamber with a potentiostat, wherein the cathode chamber and the anode chamber are separated by a semi-permeable membrane.

Embodiment 2: The method of Embodiment 1, wherein dispersing the emitted carbon dioxide in the first portion comprises dissolving, at least partially, the emitted carbon dioxide in the electrolyte solution.

Embodiment 3: The method of Embodiment 1 or 2, wherein the separating and dispersing occur within 5 minutes of each other.

Embodiment 4: The method of any one of Embodiments 1-3, wherein the biocatalyst comprises a biofilm on the cathode.

Embodiment 5: The method of any one of Embodiments 1-4, wherein the biocatalyst comprises a bacteria, archaea, fungi, algae, or any combination thereof.

Embodiment 6: The method of any one of Embodiments 1-5, wherein the emitted carbon dioxide serves as an electron acceptor to form a carbon dioxide ion.

Embodiment 7: The method of any one of Embodiments 1-6, wherein the biocatalyst reduces the emitted carbon dioxide to form the fixed carbon product.

Embodiment 8: The method of any one of Embodiments 1-7, wherein the fixed carbon product comprises methane, ethanol, or any combination thereof.

Embodiment 9: The method of any one of Embodiments 1-7, wherein the fixed carbon product comprises an organic acid.

Embodiment 10: The method of any one of Embodiments 1-9, wherein the fixed carbon product comprises a polymer.

Embodiment 11: A system comprising: a separation chamber of a gas-oil separation plant (GOSP), wherein the separation chamber is configured to separate emitted carbon dioxide from an extracted gas-oil; a GOSP carbon dioxide line for conveying the emitted carbon dioxide; a reaction chamber that contains therein electrolyte solution, wherein the electrolyte solution comprises an aqueous fluid, and wherein the reaction chamber includes therein: a cathode chamber, wherein the cathode chamber contains therein a first portion of the electrolyte solution, wherein the cathode chamber has a cathode at least partially immersed in the first portion of the electrolyte solution, wherein the cathode chamber is fluidly connected to the GOSP carbon dioxide line such that the emitted carbon dioxide is at least partially dispersed within the first portion of the electrolyte solution, wherein the cathode chamber has a biocatalyst therein, and wherein the biocatalyst is capable of converting the emitted carbon dioxide to a fixed carbon product; an anode chamber, wherein the anode chamber contains therein a second portion of the electrolyte solution, wherein the anode chamber has an anode at least partially immersed in the second portion of the electrolyte solution; a semi-permeable membrane separating the cathode chamber and the anode chamber, wherein the semi-permeable membrane conveys; and a potentiostat, wherein the potentiostat is electrically connected to the cathode and the anode, and wherein the potentiostat maintains an electric potential across the cathode and the anode.

Embodiment 12: The system of Embodiment 11, wherein the biocatalyst comprises a biofilm on the cathode.

Embodiment 13: The system of Embodiment 11 or 12, wherein the biocatalyst comprises a bacteria, archaea, fungi, algae, or any combination thereof.

Embodiment 14: The system of any one of Embodiments 11-13, wherein the emitted carbon dioxide serves as an electron acceptor to form a carbon dioxide ion.

Embodiment 15: The system of any one of Embodiments 11-14, wherein the biocatalyst reduces the emitted carbon dioxide to form the fixed carbon product.

Embodiment 16: The system of any one of Embodiments 11-15, wherein the fixed carbon product comprises methane, ethanol, or any combination thereof.

Embodiment 17: The system of any one of Embodiments 11-15, wherein the fixed carbon product comprises an organic acid.

Embodiment 18: The system of any one of Embodiments 11-17, wherein the fixed carbon product comprises a polymer.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A method comprising:
separating emitted carbon dioxide from an extracted gas-oil in a gas-oil separation chamber of a gas-oil separation plant (GOSP);
dispersing the emitted carbon dioxide in a first portion of electrolyte solution in a cathode chamber of an electrolysis reactor;
converting the emitted carbon dioxide to a fixed carbon product with a biocatalyst within the cathode chamber; and
maintaining an electric potential between a cathode of the cathode chamber and an anode of an anode chamber with a potentiostat, wherein the cathode chamber and the anode chamber are separated by a semi-permeable membrane.

2. The method of claim 1, wherein dispersing the emitted carbon dioxide in the first portion comprises dissolving, at least partially, the emitted carbon dioxide in the electrolyte solution.

3. The method of claim 1, wherein the separating and dispersing occur within 5 minutes of each other.

4. The method of claim 1, wherein the biocatalyst comprises a biofilm on the cathode.

5. The method of claim 1, wherein the biocatalyst comprises a bacteria, archaea, fungi, algae, or any combination thereof.

6. The method of claim 1, wherein the emitted carbon dioxide serves as an electron acceptor to form a carbon dioxide ion.

7. The method of claim 1, wherein the biocatalyst reduces the emitted carbon dioxide to form the fixed carbon product.

8. The method of claim 1, wherein the fixed carbon product comprises methane, ethanol, or any combination thereof.

9. The method of claim 1, wherein the fixed carbon product comprises an organic acid.

10. The method of claim 1, wherein the fixed carbon product comprises a polymer.

11. A system comprising:
a separation chamber of a gas-oil separation plant (GOSP), wherein the separation chamber is configured to separate emitted carbon dioxide from an extracted gas-oil;
a GOSP carbon dioxide line for conveying the emitted carbon dioxide;
a reaction chamber that contains therein electrolyte solution, wherein the electrolyte solution comprises an aqueous fluid, and wherein the reaction chamber includes therein:
a cathode chamber, wherein the cathode chamber contains therein a first portion of the electrolyte solution, wherein the cathode chamber has a cathode at least partially immersed in the first portion of the electrolyte solution, wherein the cathode chamber is fluidly connected to the GOSP carbon dioxide line such that the emitted carbon dioxide is at least partially dispersed within the first portion of the electrolyte solution, wherein the cathode chamber has a biocatalyst therein, and wherein the biocatalyst is capable of converting the emitted carbon dioxide to a fixed carbon product;
an anode chamber, wherein the anode chamber contains therein a second portion of the electrolyte solution, wherein the anode chamber has an anode at least partially immersed in the second portion of the electrolyte solution;
a semi-permeable membrane separating the cathode chamber and the anode chamber, wherein the semi-permeable membrane conveys; and
a potentiostat, wherein the potentiostat is electrically connected to the cathode and the anode, and wherein the potentiostat maintains an electric potential across the cathode and the anode.

12. The system of claim 11, wherein the biocatalyst comprises a biofilm on the cathode.

13. The system of claim 11, wherein the biocatalyst comprises a bacteria, archaea, fungi, algae, or any combination thereof.

14. The system of claim 11, wherein the emitted carbon dioxide serves as an electron acceptor to form a carbon dioxide ion.

15. The system of claim 11, wherein the biocatalyst reduces the emitted carbon dioxide to form the fixed carbon product.

16. The system of claim 11, wherein the fixed carbon product comprises methane, ethanol, or any combination thereof.

17. The system of claim 11, wherein the fixed carbon product comprises an organic acid.

18. The system of claim 11, wherein the fixed carbon product comprises a polymer.
